(19) United States
(12) Patent Application Publication
(10) Pub. No.: US 2015/0251008 A1
(43) Pub. Date: Sep. 10, 2015

(54) NEUROMODULATORY DEVICES, SYSTEMS, AND METHODS FOR TREATING FIBROMYALGIA

(71) Applicant: Ohio State Innovation Foundation, Columbus, OH (US)

(72) Inventors: Ali R. Rezai, Columbus, OH (US); Milind Deogaonkar, Broadview Heights, OH (US)

(21) Appl. No.: 14/643,262

(22) Filed: Mar. 10, 2015

Related U.S. Application Data
(60) Provisional application No. 61/950,418, filed on Mar. 10, 2014.

(51) Int. Cl. A61N 1/36; A61N 7/00; A61N 1/40

(52) U.S. Cl. CPC A61N 1/36071; A61N 1/36053; A61N 1/403; A61N 1/36139; A61N 7/00; A61N 2007/0026

(57) ABSTRACT

One aspect of the present disclosure relates to a method for treating fibromyalgia. One step of the method can include placing a therapy delivery device into electrical communication with a parasympathetic nervous system (PNS) nerve target, other than the vagus serve, which is associated with the fibromyalgia. Next, the therapy delivery device cars he activated to deliver a therapy signal to the PNS nerve target in an amount and for a time sufficient to effect a change m parasympathetic activity in the subject and thereby treat the fibromyalgia.

### RELATED APPLICATION

This application claims the benefit of U.S. Provisional Patent Application Ser. No. 61/950,418, filed Mar. 10, 2014, the entirety of which is hereby incorporated by reference for all purposes.

### TECHNICAL FIELD

The present disclosure relates generally to neuromodulatory devices, systems and methods, and more particularly to devices, systems, and methods for treating fibromyalgia.

### BACKGROUND

Fibromyalgia is a chronic disorder characterized by widespread musculoskeletal pain and tenderness to palpation at specific tender points, in addition, fibromyalgia patients often describe other symptoms such as fatigue, sleep disturbances, headache or cognitive dysfunction, associated with the syndrome. The American College of Rheumatology has defined fibromyalgia as pain in all four quadrants combined with axial skeletal pain, and at least 11 of 18 tender point sites. Widespread pain must have been present for at least 3 months. Tender points, the diagnostic hallmark of fibromyalgia, are examples of hyperalgesia, thought to be due to central sensitization. Patients with fibromyalgia have quantitatively altered perception compared to pain-free patients, suggesting that people with fibromyalgia process sensory information differently.

Patients often report widespread pain over all parts of the body which often seems to arise in the muscles. The pain shows varying intensities that wax and wane over time, it is profound, widespread and chronic, and is often severely debilitating, having profound effects on the quality of life of the patients. Typically, the pain is described as deep muscular aching, throbbing, twitching, stabbing and shooting pain. Neurological complaints such as numbness, tingling and burning are often present. The severity of the pain and stiffness is often, worse in the morning. Aggravating factors that affect pain include cold/humid weather, non-restorative sleep, physical and mental fatigue, excessive physical, activity, physical inactivity, anxiety and stress. Additionally to pain, patients commonly complain of fatigue in form of an all-encompassing exhaustion that interferes with even the simplest daily activities. Within the spectrum of symptoms are a decreased sense of energy, disturbances of sleep, problems with memory and concentration and varying degrees of anxiety and depression.

Certain other medical conditions are sometimes associated with fibromyalgia, such as tension headaches, migraine, irritable bowel syndrome, overactive bladder, pelvic pain, premenstrual tension syndrome, cold intolerance, dry eyes and mouth, anxiety, depression, ringing in the ears, dizziness, vision problems and others. Patients with established rheumatoid arthritis, lupus (SLE) and Sjogren's syndrome often develop fibromyalgia symptoms during the course of their disease. The complexity of the syndrome, with multiple and highly diverse symptoms described by the patients has meant that effective and long-term relief above all of the pain, has proved elusive. Common analgesics have limited effectiveness, especially over the long-term.

The present disclosure relates generally to neuromodulator devices, systems and methods, and more particularly to devices, systems, and methods for treating fibromyalgia.

One aspect of the present disclosure relates to a method for treating fibromyalgia. One step of the method can include placing a therapy delivery device into electrical communication with a parasympathetic nervous system (PNS) nerve target, other than the vagus nerve, which is associated with the fibromyalgia. Next, the therapy delivery device can be activated to deliver a therapy signal to the PNS nerve target hi an amount and for a time sufficient to effect a change in parasympathetic activity in the subject and thereby treat the fibromyalgia.

Another aspect of the present disclosure relates to a method for treating fibromyalgia. One step of the method can include placing a therapy delivery device into electrical communication with an autonomic nervous system (ANS) nerve target associated with the fibromyalgia. Next, the therapy delivery device can be activated to deliver an ultrasonic therapy signal to the ANS nerve target in an amount and for a time sufficient to effect, a change in parasympathetic and/or sympathetic activity in the subject and thereby treat the fibromyalgia,

Another aspect of the present disclosure relates to a method for treating fibromyalgia. One step of the method can include placing a therapy delivery device into electrical communication with an ANS nerve target associated with the fibromyalgia. Next, the therapy delivery device can be activated to deliver a radio-frequency therapy signal to the ANS nerve target in an amount and for a time sufficient to effect a change in parasympathetic and/or sympathetic activity in the subject and thereby treat the fibromyalgia.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a schematic illustration showing the cervical and upper thoracic portions of the sympathetic nerve chain and the spinal cord;

FIG. 2 is a schematic illustration showing a transcutaneous neuromodulatory device constructed in accordance with another aspect of the present disclosure;

FIGS. 3A-B are schematic illustrations showing alternative transcutaneous neuromodulatory devices constructed in accordance with other aspects of the present disclosure; and

FIG. 4 is a process flow diagram illustrating a method for treating fibromyalgia in a subject according to another aspect of the present disclosure.

### DETAILED DESCRIPTION

Definitions

Unless defined otherwise, all technical and scientific terms used herein have the same meaning as is commonly understood by one of skill in the art to which the present disclosure pertains.

In the context of the present disclosure, the term “autonomic nervous tissue” can refer to any tissues of the sympathetic nervous system (SNS) or the parasympathetic nervous system (PNS) including, but not limited to, neurons, axons, fibers, tracts, nerves, plexus, afferent plexus fibers, efferent plexus fibers, ganglia, pre-ganglionic fibers, postganglionic fibers, afferents, efferents, and combinations thereof In some instances, autonomic nervous tissue, ears comprise an autonomic nervous system (ANS) nerve target.

As used herein, the term “sympathetic nervous tissue” can refer to any tissues of the sympathetic nervous system (SNS) including, but not limited to, neurons, axons, fibers, tracts, nerves, plexus, afferent plexus fibers, efferent plexus fibers, ganglia, pre-ganglionic fibers, post-ganglionic fibers, cervical ganglia/ganglion, a cervicothoracic or stellate ganglion, thoracic ganglia/ganglion, afferents, efferents, and combinations thereof. In some instances, sympathetic nervous tissue can comprise a SNS nerve target, in some instances, the term can also refer to peripheral sympathetic nervous tissue.

As used herein, the term “central nervous system” can refer to the part of the nervous system consisting of the brain and spinal cord.

As used herein, the phrase “spinal cord stimulation” can refer to stimulation of any spinal nervous tissue (e.g., a spinal cord segment), including spinal neurons, accessory neuronal cells, nerves, nerve roots, nerve fibers, or tissues that are associated with the-spinal cord.

As used herein, the phrase “spinal nervous tissue” can refer to nerves, neurons, neuroglial cells, glial cells, neuronal accessory cells, nerve roots, nerve fibers, nerve rootlets, parts of nerves, nerve bundles, mixed nerves, sensory fibers, motor fibers, dorsal root, ventral root, dorsal root ganglion, spinal ganglion, ventral motor root, general somatic afferent fibers, general visceral afferent fibers, general somatic efferent fibers, general visceral efferent fibers, grey matter, white matter, the dorsal column, the lateral column, and/or the ventral column associated with the spinal cord.

As used herein, the term “fibromyalgia” can refer to chronic and frequently difficult-to-manage pain in muscle and soft tissues surrounding the joints of unknown, etiology characterized by widespread pain, abnormal pain processing, sleep disturbance, fatigue and psychological distress. In some instances, the terms “fibromyalgia,” “fibromyalgia syndrome,” “myofascial syndrome” and “fibromyalgia and related syndromes” can be used interchangeably and refer to a chronic pain disorder characterized by one or more of the following: pain including allodynia (painful response to a stimulus that is not usually painful) and hyperalgesia (exaggerated response to a stimulus that is usually only mildly painful); a series of regional pains, such as non-cardiac chest pain, dyspepsia, headache, abdominal cramping (irritable bowel syndrome), temporomandibular pain and chronic pelvic pain; stiffness; chronic aching in multiple areas of the musculoskeletal system; fatigue; poor sleep; tender points; cognitive difficulties with attention and memory: weight fluctuations; allergic symptoms (e.g., nasal congestion); hypersensitivity to environmental stimuli (e.g., odors, bright lights, loud noises) and medications; syncope; shortness of breath; and urinary frequency and urgency. In other instances, the 2010 criteria set forth by the American College of Rheumatology can be used to diagnose and/or assess severity of fibromyalgia in a subject. For instance, diagnosis can be based on: a Widespread Pain Index (WPI) >7 and a symptom severity scale (SS) >5 or WPI 3-6 and SS >9; symptoms having been present at a similar level for at least 3 months; and/or the subject, not having a disorder that would otherwise explain the pain. The term “fibromyalgia” can also refer to primary or secondary fibromyalgia. “Primary fibromyalgia” can refer to fibromyalgia in which the only rheumatic disorder the subject is suffering from is fibromyalgia, whereas the term, “secondary fibromyalgia” can refer to fibromyalgia that occurs in conjunction with another diagnosed rheumatic disorder. In some instances, “fibromyalgia and related syndromes” can include fibromyalgia-related fatigue syndromes, pain syndromes, and sleep disturbances.

As used herein, the terms “modulate” or “modulating” with reference to autonomic nervous tissue can refer to causing a change in neuronal activity, chemistry, and/or metabolism. The change can refer to an increase, decrease, or even a change in a pattern of neuronal activity. The terms may refer to either excitatory or inhibitory stimulation, or a combination thereof, and may be at least electrical, magnetic, optical or chemical, or a combination of two or more of these. The terms “modulate” or “modulating” can also be used to refer to a masking, altering, overriding, or restoring of neuronal activity.

As used herein, the term “intraluminal target site” can refer to a desired anatomical location at which a therapy delivery device may be positioned. The intraluminal target site can comprise a variety of locations, including intraluminal and extraluminal locations innervated by, or in electrical communication with, nervous tissue. In one example, an intraluminal target site can comprise an intravascular location in electrical communication with at least one nerve of the ANS. Intraluminal target sites contemplated by the present disclosure are described in further detail below.

As used herein, the term “electrical communication” can refer to the ability of an electric field generated by an electrode or electrode array to be transferred, or to have a neuromodulatory effect, within and/or on at least one nerve, neuron, and/or nervous tissue (e.g., of the ANS).

As used herein, the term “in communication” can refer to at least a portion of a therapy delivery device or therapy delivery system being adjacent, in the general vicinity, in close proximity, or directly next to and/or directly on an ANS nerve target. In some instances, the term can mean that at least a portion of a therapy delivery device or therapy delivery system is “in communication” with an ANS nerve target if application of a therapy signal (e.g., an electrical and/or chemical signal) thereto results in a modulation of neuronal activity to elicit a desired response, such as modulation of a sign or symptom associated with fibromyalgia.

As used herein, the term “subject” can be used interchangeably with the term “patient” and refer to any warm-blooded organism including, but not limited to, human beings, pigs, rats, mice, dogs, goats, sheep, horses, monkeys, apes, farm animals, livestock, rabbits, cattle, etc.

As used herein, the terms “substantially blocked” or “substantially block” when used with reference to autonomic nervous tissue activity can refer to a complete (e.g., 100%) or partial inhibition (e.g., less than 100%, such as about 90%, about 80%, about 70%, about 60%, or less than about 50%) of nerve conduction through the nervous tissue.

As used herein, the term “activity” when used with reference to autonomic nervous tissue can, in some instances, refer to the ability of a nerve, neuron, or fiber to conduct, propagate, and/or generate an action potential. In other instances, the term can refer to the frequency at winch a nerve or neuron is conducting, propagating, and/or generating one or more action potentials at a given moment in time. In further instances, the term can refer to the frequency at which a nerve or neuron is conducting, propagating, and/or generating one or more action potentials over a given period of time (e.g., seconds, minutes, hours, days, etc.).

As used herein, the terms “treat” or “treating” can refer to therapeutically regulating, preventing, improving, alleviating the symptoms of, and/or reducing the effects of fibromyalgia and symptoms associated therewith.

As used herein, the singular forms “a,” “an” and “the” can include the plural forms as well, unless the context clearly indicates otherwise. It will, be further understood that the terms “comprises” and/or “comprising,” as used herein, can specify the presence of stated features, steps, operations, elements, and/or components, but do not preclude the presence or addition of one or more other features, steps, operations, elements, components, and/or groups thereof.

As used herein, the term “and/or” can include any and all combinations of one or more of the associated listed items.

As used herein, phrases such as “between X and Y” and “between about X and Y” can be interpreted to include X and Y.

As used herein, phrases such as “between about X and Y” can mean “between about X and about Y.”

As used herein, phrases such as “from about X to Y” can mean “from about X to about Y.”

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “directly adjacent” another feature may have portions that overlap or underlie the adjacent feature, whereas a structure or feature that is disposed “adjacent” another feature may not have portions that overlap or underlie the adjacent feature.

Spatially relative terms, such as “under,” “below,” “lower,” “over,” “upper” and the like, may be used herein for ease of description to describe one element or feature's relationship to another element(s) or feature(s) as illustrated in the figures. It will be understood that the spatially relative terms can encompass different orientations of a device in use or operation, in addition to the orientation depicted in the figures. For example, if a device in the figures is inverted, elements described as “under” or “beneath” other elements or features would then be oriented “over” the other elements or features.

It will be understood that, although the terms “first,” “second,” etc. may be used herein to describe various elements, these elements should not be limited by these terms. These terms are only used to distinguish one element from another. Thus, a “first” element discussed below could also be termed a “second” element without departing from the teachings of the present disclosure. The sequence of operations (or steps) is not limited to the order presented in the claims or figures unless specifically indicated otherwise.

When an element or structure is referred to as being “on,” “engaged to,” “connected to,” or “coupled to” another element or structure, it may be directly on, engaged, connected or coupled to the other element or structure, or intervening elements or structures may be present. In contrast, when an element Is referred to as being “directly on,” “directly engaged to,” “directly connected to,” or “directly coupled to” another element or structure, there may be no intervening elements or structures present. Other words used, to describe the relationship between elements should be interpreted in a like fashion (e.g., “between” versus “directly between,” “adjacent” versus “directly adjacent,” etc.)

Overview

A brief discussion of the pertinent neurophysiology is provided to assist the reader with understanding certain aspects of the present disclosure.

The nervous system is divided into the somatic nervous system and the ANS. In general, the somatic nervous system controls organs under voluntary control (e.g., skeletal muscles) and the ANS controls individual organ function and homeostasis. For the most part, the ANS is not subject to voluntary control. The ANS is also commonly referred to as the visceral or automatic system.

The ANS can be viewed as a “real-time” regulator of physiological functions which extracts features from the environment and, based on that information, allocates an organism's internal resources to perform physiological functions for the benefit of the organism, e.g., responds to environment conditions in a manner that is advantageous to the organism.

The ANS conveys sensory impulses to and from the central nervous system to various structures of the body such as organs awl blood vessels, in addition to conveying sensory impulses through reflex arcs. For example, the ANS controls; constriction and dilatation of blood vessels; heart rate; the force of contraction of the heart; contraction and relaxation of smooth muscle in various organs; lungs; stomach; colon; bladder; visual accommodation; and secretions from exocrine and endocrine glands, etc. The ANS does this through a series of nerve fibers, and more specifically through efferent and afferent nerves. The ANS acts through a balance of its two components: the sympathetic nervous system (SNS) and the parasympathetic nervous system (PNS), which are two anatomically and functionally distinct systems. Both of these systems include myelinated preganglionic fibers which make synaptic connections with unmyelinated postganglionic fibers, and it is these fibers which then innervate the effector structure. These synapses usually occur in clusters called ganglia. Most organs are innervated by fibers from both divisions of the ANS, and the influence is usually opposing (e.g., the vagus nerve slows the heart, while the sympathetic nerves increase its rate and contractility), although it may be parallel (e.g., as in the case of the salivary glands). Each of these is briefly reviewed below.

The PNS is the part of the ANS controlling a variety of autonomic functions including, but not. limited to, involuntary muscular movement of blood vessels and gut and glandular secretions from eye, salivary glands, bladder, rectum and genital organs. The vagus nerve is part of the PNS. Parasympathetic nerve fibers are contained within the last five cranial nerves and the last three spinal nerves and terminate at parasympathetic ganglia near or in the organ they supply. The actions of the PNS are broadly antagonistic to those of the SNS lowering blood pressure, slowing heartbeat, stimulating the process of digestion etc. The chief neurotransmitter in the PNS is acetylcholine. Neurons of the parasympathetic nervous system emerge from the brainstem as part of the Cranial nerves III, VII, IX and X (vagus nerve) and also from the sacral region of the spinal cord via Sacral nerves. Because of these origins, the PNS is often referred to as the “craniosacral outflow”.

In the PNS, both pre- and post-ganglionic neurons are cholinergic (i.e., they utilize the neurotransmitter acetylcholine). Unlike adrenaline and noradrenaline, which the body takes around 90 minutes to metabolize, acetylcholine is rapidly broken down after release by the enzyme cholinesterase. As a result the effects are relatively brief in comparison to the SNS,

Each pre-ganglionic parasympathetic neuron synapses with just a few postganglionic neurons, which are located near, or in, the effector organ, a muscle or gland. As noted above, the primary neurotransmitter in the PNS is acetylcholine such that acetylcholine is the neurotransmitter at all the pre- and many of the post-ganglionic neurons of the PNS. Some of the postganglionic neurons, however, release nitric oxide as their neurotransmitter.

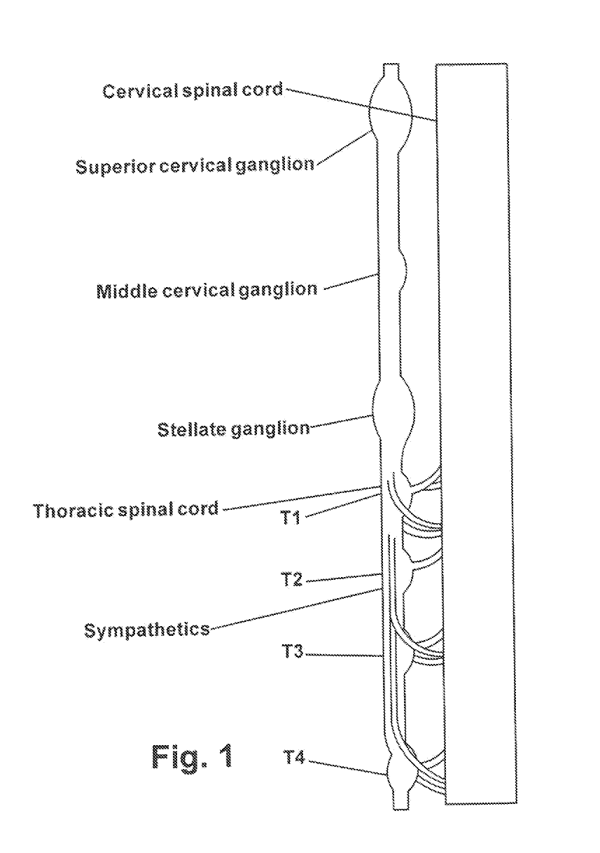

The SNS is the part of the ANS comprising nerve fibers that leave the spinal cord in the thoracic and lumbar regions and supply viscera and blood vessels by way of a chain of sympathetic ganglia running on each side of the spinal column which communicate with the central nervous system via a branch to a corresponding spinal nerve. The sympathetic trunks extend from the base of the skull to the coccyx. The cephalic end of each is continued upward through the carotid canal into the skull, and forms a plexus on the internal carotid artery; the caudal ends-of the trunks converge and end in a single ganglion, the ganglion impar, placed in front of the coccyx. As partly shown in FIG. 1, the ganglia of each trunk are distinguished as cervical, thoracic, lumbar, and sacral and, except in the neck, they closely correspond in number to the vertebrae.

The SNS controls a variety of autonomic functions including, but not limited to, control of movement and secretions from viscera and monitoring their physiological state, stimulation of the sympathetic system inducing, e.g., the contraction of gut sphincters, heart muscle and the muscle of artery walls, and the relaxation of gut smooth muscle and the circular muscles of the iris. The chief neurotransmitter in the SNS is adrenaline, which is liberated m the heart, visceral muscle, glands and internal vessels, with acetylcholine acting as a neurotransmitter at ganglionic synapses and at sympathetic terminals in skin and skeletal muscles. The actions of the SNS tend to be antagonistic to those of the PNS.

The neurotransmitter released by the post-ganglionic neurons is noradrenaline (also called norepinephrine). The action of noradrenaline on a particular structure, such as a gland or muscle, is excitatory in some cases and inhibitory in others. At excitatory terminals, ATP may be released along with noradrenaline. Activation of the SNS may be characterized as general because a single pre-ganglionic neuron usually synapses with many postganglionic neurons, and the release of adrenaline from the adrenal medulla into the blood ensures that ail the cells of the body will be exposed to sympathetic stimulation even if no postganglionic neurons reach them directly.

The present disclosure relates generally to neuromodulatory devices, systems and methods, and more particularly to devices, systems, and methods for treating fibromyalgia. The ANS plays a crucial role in the function and regulation of the peripheral cardiac, gastric, vascular and immunological tissues, as well as other related tissues. Neuromodulation of the ANS is a precise, controlled, and highly targeted approach to influence and impact the function and dysfunction in humans. Neuromodulation according to the present disclosure can improve the function, activate, inhibit, modulate, and impact the intrinsic autonomic tone, such as sympathetic and/or parasympathetic activity, the dysregulation or imbalance of which may be associated with (e.g., causative of) fibromyalgia. As described in detail below, the present disclosure can advantageously provide, in some instances, devices, systems, and methods for uncoupling dysfunctional nerve signals from the brain to the ANS (as well as ascending signals into the CNS), as well as dysfunctional nerve signals from the ANS to peripheral tissues (e.g., peripheral cardiac, gastric, vascular and immunological tissues) to effectively normalize or regulate the ANS. By employing such devices, systems and methods, the present disclosure can effectively prevent, mitigate, and/or treat fibromyalgia.

Therapy Delivery Devices

In one aspect, the present disclosure includes various therapy delivery devices (not shown) and related systems configured to treat fibromyalgia in a subject. In some instances, therapy delivery devices that may be used to practice the present disclosure may be positioned directly on an ANS nerve target, neuron or nerve structure. In other instances, therapy delivery devices that may be used to practice the present disclosure may be positioned below the skin of a subject but not directly on an ANS nerve target, neuron or nerve structure. In further instances, therapy delivery devices that may be used to practice the present disclosure may comprise an external device, e.g., positioned in a lumen adjacent an ANS nerve target, neuron or nerve structure. In still further instances, therapy delivery devices used to practice the present disclosure can include an external device, e.g., positioned on the skin of a subject adjacent an ANS nerve target neuron or nerve structure. Therapy delivery devices can be temporarily or permanently implanted within, on, or otherwise associated with a subject suffering from, afflicted by, or suspected of having fibromyalgia.

Therapy delivery devices of the present disclosure can be configured to deliver various types of therapy signals to an ANS nerve target, neuron or nerve structure. For example, therapy delivery devices of the present disclosure can be configured to deliver only electrical energy, only ultrasound, only radiofrequency, only microwave, only magnetic, only a pharmacological or biological agent (e.g., gene therapy), or a combination thereof. In one example, therapy delivery devices of the present disclosure can comprise at least one electrode and an integral or remote electrical energy generator (not shown), which is in electrical communication with the one or more electrodes and configured to produce one or more electrical signals (or pulses). In another example, therapy delivery devices can include a pharmacological or biological agent reservoir, a pump, and a fluid dispensing mechanism. Non-limiting examples of pharmacological and biological agents can include chemical compounds, drugs, nucleic acids, polypeptides, stem cells, toxins (e.g., botulinum), as well as various energy forms, such as ultrasound, radiofrequency (continuous or pulsed), magnetic waves, cryotherapy, and the like. Energy (stimulation signals) can he delivered to ANS nerve targets continuously, intermittently, cyclically, or as part of a linkage or in association with internal and/or external body signals (e.g., sensed physiological parameters). In yet another example, therapy delivery devices can be configured to deliver magnetic nerve stimulation with desired field locality and depth of penetration. One skilled in the art will appreciate that combinations of the therapy delivery devices above configurations are also included within the scope of the present disclosure.

In some instances, therapy delivery devices can include a neurostimulator (or inhibitor), such as an electrode, a controller or programmer, and one or more connectors for connecting the stimulating (or inhibiting) device to the controller, in further describing representative electrodes, which are described in the singular, it will be apparent that more than one electrode may be used as part of a therapy delivery device. Accordingly, the description of a representative electrode suitable for use in the therapy delivery devices of the present disclosure is applicable to other electrodes that may be employed.

An electrode can be controllable to provide output signals that may be varied in voltage, frequency, pulse-width, current and intensity. The electrode can also provide both positive and negative current flow from the electrode and/or is capable of stopping current flow from the electrode and/or changing the direction of current flow from the electrode. In some instances, therapy delivery devices can include an electrode that is controllable, i.e., in regards to producing positive and negative current flow from the electrode, stopping current flow from the electrode, changing direction of current flow from the electrode, and the like. In other instances, the electrode has the capacity for variable output, linear output and short pulse-width. The electrode can have any suitable geometry, such as cylindrical, flat or split thickness.

The electrical energy generator can comprise a battery or generator, such as a pulse generator that is operatively connected to the electrode. For example, the electrical energy generator can include a battery that is rechargeable by inductive coupling. The electrical energy generator may he positioned in any suitable location, such as adjacent the electrode (e.g., implanted adjacent the electrode), or a remote site in or on the subject's body or away from the subject's body in a remote location. An electrode may be connected to the remotely positioned electrical energy generator using wires, e.g., which may be implanted at a site remote from the electrode or positioned outside the subject's body. In one example, implantable electrical energy generators analogous to a cardiac pacemaker may be used.

The electrical energy generator can control the pulse waveform, the signal pulse width, the signal pulse frequency, the signal pulse phase, the signal pulse polarity, the signal pulse amplitude, the signal pulse intensity, the signal pulse duration, and combinations thereof of an electrical signal. The electrical energy generator may be used to convey a variety of currents and voltages to one or more electrodes and thereby modulate the activity of an ANS nerve, neuron, or nerve structure. The electrical energy generator may be used to control numerous electrodes independently or in various combinations as needed to provide stimulation. In some instances, an electrode may be employed that includes its own power source, which is capable of obtaining sufficient power for operation from surrounding tissues in the subject's body, or which may be powered by bringing a power source external to the subject's body into contact with the subject's skin, or which may include an integral power source.

In other instances, an electrical signal may be constant, varying and/or modulated with respect to the current, voltage, pulse-width, cycle, frequency, amplitude, and so forth. For example: a current may range from about 0.001 microampere (mA.) to about 1000 mA and, more specifically, from about 0.1 mA to about 100 mA; a voltage may range from about 0.1 millivolt to about 25 volts; a frequency may range from about 0.5 to about 10,000 Hz; and a pulse-width may range from about 10 microseconds to about 10,000 microseconds. The type of stimulation may vary and involve different waveforms known to the skilled artisan (e.g., square, sinus, etc.). for example, the stimulation maybe based on the H waveform found in nerve signals (i.e., Hoffman Reflex). In another example, different forms of interferential stimulation may be used.

To increase activity in a portion of the ANS, for example, voltage or intensity may range from about 1 millivolt to about 1volt or more, e.g., 0.1 volt to about 50 volts (e.g., from about 0.2 volt to about 20 volts), and the frequency may range from about 1 Hz to about 2500 Hz, e.g., about 1 Hz to about 1000 Hz (e.g., from about 2 Hz to about 100 Hz). In some instances, pure DC and/or AC voltages may be employed. The pulse-width may range from about 1microsecond to about 2000 microseconds or more, e.g., from about 10 microseconds to about 2000 microseconds (e.g., from about 15 microseconds to about 1000 microseconds). The therapy signal may be applied for at least about 1 millisecond or more, e.g., about 1 second (e.g., about several seconds). In some instances, stimulation may be applied for as long as about 1 minute or more, e.g., about several minutes or more (e.g., about 30 minutes or more).

To decrease activity in a portion of the ANS, for example, voltage or intensity may range from about 1 millivolt to about 1 volt, or more, e.g., 0.1 volt to about 50 volts (e.g., from about 0.2 volt to about 20 volts), and the frequency may range from about 1 Hz to about 2500 Hz, e.g., about 50 Hz to about 2500 Hz. In some instances, pure DC and/or AC voltages may be employed. The pulse-width may range from about 1 microseconds to about 2000 microseconds or more, e.g., from about 10 microseconds to about 2000 microseconds (e.g., from about 15 microseconds to about 1000 microseconds). The therapy signal may be applied for at least about. 1 millisecond or more, e.g., about 1 second (e.g., about several seconds). In some instances, the electrical energy may be applied for as long as about 1 minute or more, e.g., about several minutes or more (e.g., about 30 minutes or more may be used).

The electrode may be mono-polar, bipolar or multi-polar. To minimize the risk of an immune response triggered by the subject against the therapy delivery device, and also to minimize damage thereto (e.g., corrosion from other biological fluids, etc.), the electrode (and any wires and optional housing materials) can be made of inert materials, such as silicon, metal, plastic and the like. In one example, a therapy delivery device can include a multi-polar electrode having about four exposed contacts (e.g., cylindrical contacts).

A controller or programmer may also be associated with a therapy delivery device. A programmer, for example, can include one or more microprocessors under the control of a suitable software program. Other components of a programmer, such as an analog-to-digital converter, etc., will be apparent to those of skill in the art.

Therapy delivery devices can be pre-programmed with desired stimulation parameters. Stimulation parameters can be controllable so that a therapy signal may be remotely modulated to desired settings without removal of the electrode from its target position. Remote control may be performed, e.g., using conventional telemetry with an implanted signal generator and battery, an implanted radiofrequency receiver coupled to an external transmitter, and the like. In some instances, some or all parameters of the electrode may be controllable by the subject, e.g., without supervision by a physician. In other instances, some or all parameters of the electrode may be automatically controllable by a programmer or controller comprising the therapy delivery device.

In one example, the therapy delivery device can he configured for percutaneous placement or implantation, in this instance, the therapy delivery device can comprise one or more implantable electrodes shaped or configured, for example, as a wire, a rod, a filament, a ribbon, a cord, a tube, a formed wire, a flat strip, or a combination thereof. In one example, one or more of the electrodes can comprise a laminotomy electrode array. Laminotomy electrodes, for example, generally have a flat paddle configuration and typically possess a plurality of electrodes (e.g., 2, 3, 4 or more) arranged on the paddle. The arrangement of electrodes on the paddle may be in rows and columns, staggered, spaced, circular, or any other arrangement that will position the electrodes for optimal delivery of electrical energy. In another example, a therapy delivery device configured for percutaneous implantation can include an electrode configured to attach itself to a deep-seated ANS nerve target, neuron, or nerve structure (e.g., a ganglion). In such instances, the self-attaching electrode advantageously does not require sutures for attachment to the ANS nerve target, neuron, or nerve structure, nor does the electrode require open surgery for implantation. One or more implantable electrodes may be controlled individually, in series, in parallel, or any other manner desired. Once implanted, the implantable eleetrode(s) may be held in position using any method known to the skilled artisan, such as stitches, epoxy, tape, glue, sutures, or a combination thereof.

In another example, the therapy delivery device can be configured for intravascular or Intraluminal placement or implantation. In some instances, a therapy delivery device configured for intravascular or intraluminal placement or implantation can be configured in an identical or similar manner as the expandable electrode disclosed in U.S. patent application Ser. No. 11/641,331 to Greenberg et al. thereinafter, “the '331 application”).

In yet another example, the therapy delivery device can be configured for transcutaneous neuromodulation. In some instances, transcutaneous neuromodulation can include positioning an electrode on a skin surface so that a therapy signal can be delivered to an ANS nerve target, neuron, or nerve structure. Transcutaneous neuromodulation can additionally include partially transcutaneous methods (e.g., using a fine, needle-like electrode to pierce the epidermis). In other instances, a surface electrode can be placed into electrical contact with an ANS nerve target, neuron, or nerve structure (e.g., of the ANS) associated with fibromyalgia. For example, a surface electrode can he placed on a target zone of the skin that overlies an ANS nerve target (e.g., a stellate ganglion). Generally, a therapy signal (e.g., electrical energy) used for transcutaneous neuromodulation may be constant, varying and/or modulated, with respect to the current, voltage, pulse-width, cycle, frequency, amplitude, and so forth (e.g., the current may be between about 1 to 100 microampere), about 1.0 V (average), about 1 to about 1000 Hz, with a pulse-width of about 250 to about 500 microseconds.

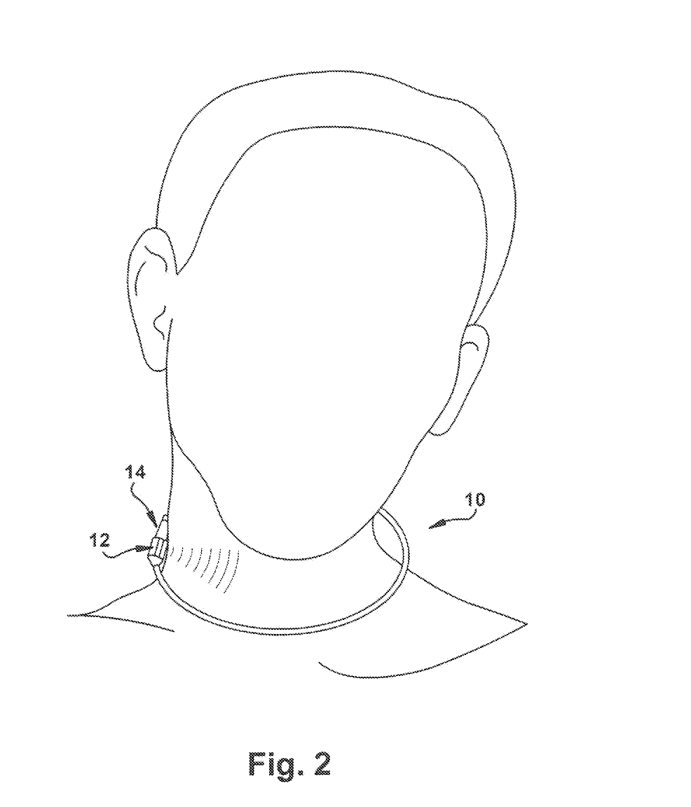

In one example, a transcutaneous neuromodulation device can comprise a wearable accessory item, such as a necklace or collar 10 (FIG. 2). As shown in FIG. 2, a necklace or collar 10 can be configured to include at least, one electrode 12 for delivering a therapy signal to a particular region of a subject's neck (e.g., an anterior or posterior region thereof) depending upon the desired neuromodulatory effect. The necklace or collar 10 can additionally include an integral power source 14 (e.g., a rechargeable battery), it will be appreciated that the electrode(s) 12 can alternatively be powered by a wireless power source (not shown). The necklace or collar 10 can be configured to obtain a pre-selected position about a subject's neck by, for example, using a positioning guide (not shown), weighting the necklace or collar, etc. Alternatively, the subject can manually adjust the necklace or collar 10 as needed to optimize delivery of the therapy signal from the electrode(s) 12 to the nerve target. Other examples of wearable accessory items that can be configured as a transcutaneous neuromodulation device include pendants, buttons, earrings, etc.

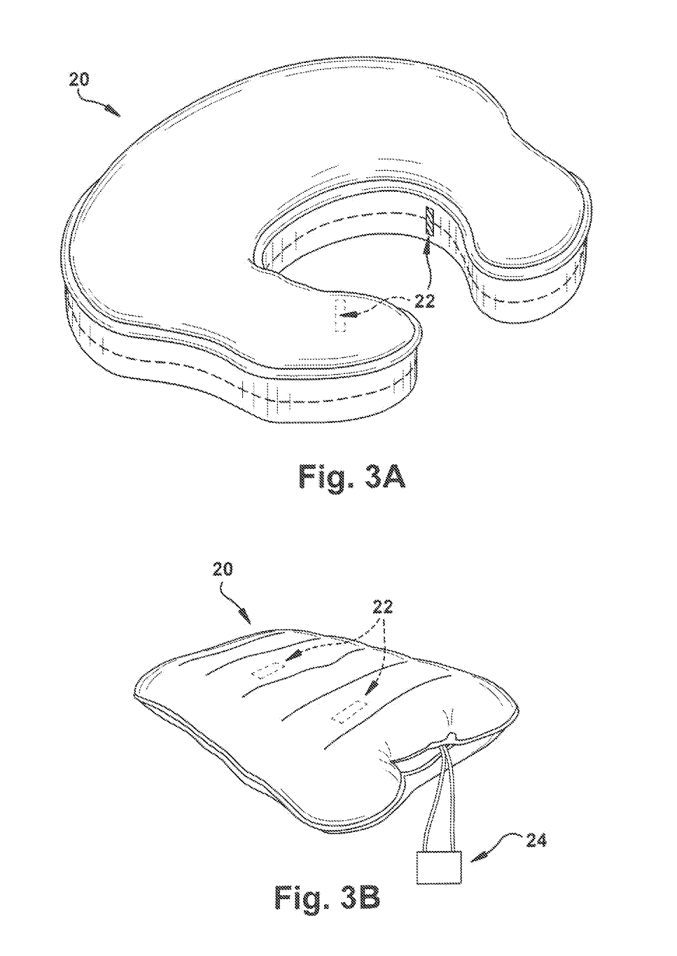

In another example, a transcutaneous neuromodulation device can comprise a pillow 20 (FIGS. 3A-B). In some instances, the pillow 20 (FIG. 3A) can be configured as a collar for use in a reclined or upright position, such as on an airplane, in a car, on a couch, etc. The pillow 20 can include at least one electrode 22 configured to deliver a therapy signal to an ANS nerve target (e.g., in a subject's head or neck). In other instances, the electrode 22 can comprise a coil configured to deliver magnetic stimulation. As shown in FIG. 3A, the pillow 20 includes two oppositely disposed electrodes 22. The pillow 20 can also include a power source (not shown), which may be integrally connected with the pillow or located remotely (i.e., wirelessly) therefrom. In other instances, the pillow 20 (FIG. 3B) can comprise a traditional or conventional pillow for use when a subject is sleeping or lying in bed. As shown in FIG. 3B, the pillow 20 can include two oppositely disposed electrodes 22 configured to deliver a therapy signal to an ANS nerve target when the subject's neck or head is straddled between the electrodes. The pillow 20 can further include a power source 24 that is in direct electrical communication with the electrodes 22; however, it will be appreciated that the power source can be located remotely (i.e., wirelessly) from the pillow.

It will be appreciated that the transcutaneous neuromodulation devices illustrated in FIGS. 2-3B are illustrative only and, moreover, that such devices can include any wearable item, accessory, article of clothing, or any object, device, or apparatus that a subject can use and, during use, comes into close or direct contact with a portion of the subject's body (e.g., the subject's neck). Examples of such transcutaneous neuromodulation devices can include vests, sleeves, shirts, socks, shoes, underwear, belts, scarves, wrist bands, gloves, ear pieces, band-aids, turtle neck, pendants, buttons, earrings, stickers, patches, bio-films, skin tattoos (e.g., using neuro-paint), chairs, computers, beds, head rests (e.g., of a chair or car seat), cell phones, and the like, it will also be appreciated that the transcutaneous neuromodulation devices can include other components as described above, such as a controller (e.g., configured to automatically coordinate operation of the power source) and at least one sensor for detecting a physiological parameter of interest.

Therapy delivery devices can be part of an open- or closed-loop system. In an open-loop system, for example, a physician or subject may, at any time, manually or by the use of pumps, motorized elements, etc., adjust treatment parameters, such as pulse amplitude, pulse-width, pulse frequency, duty cycle, dosage amount, type of pharmacological or biological agent, etc. Alternatively, in a closed-loop system, treatment parameters (e.g., electrical signals) may be automatically adjusted in response to a sensed physiological parameter or a related symptom indicative of the extent and/or presence of fibromyalgia (or a symptom thereof). In a closed-loop feedback system, a sensor (not shown) that senses a physiological parameter associated with fibromyalgia can be utilized. More detailed descriptions of sensors that may be employed in a closed-loop system, as well as other examples of sensors and feedback control techniques that may be employed as part of the present disclosure are disclosed in U.S. Pat. No. 5,716,377.

It should be appreciated that incorporating a therapy delivery device as part of a closed-loop system, can include placing or implanting a therapy delivery device on or within a subject at an ANS nerve target, sensing a physiological parameter associated with fibromyalgia, and then activating the therapy delivery device to apply a therapy signal to adjust application of the therapy signal to the ANS nerve target in response to the sensor signal to treat fibromyalgia. In some instances, such physiological parameters can include any characteristic or function associated with fibromyalgia, such as the activity of an ANS nerve or nerve structure (e.g., sympathetic ganglia, afferent and efferent nerves branches thereof, the spinal cord, and upstream/downstream connections), protein concentrations, electrochemical gradients, hormones (e.g., Cortisol), neuroendocrine markers, electrolytes, laboratory values, vital signs (e.g., blood pressure), markers of locomotor activity, cardiac markers (e.g., EKG RR intervals), or other signs and biomarkers associated with fibromyalgia.

In one example of a closed-loop system, one or more sensors can be used to detect the intrinsic tone and activity of certain ANS nerve targets (e.g., one or more sympathetic ganglia and/or parasympathetic nerves) at all stages of fibromyalgia and its manifestations. A controller associated with the closed-loop system can be configured to store and record the detected intrinsic tone and activity. If detected activity is reduced, increased or altered, the system can modulate the activity of the ANS on demand (e.g., manually) or automatically (e.g., continuously). For example, if the activity is too high (e.g., indicative of a hypersympathetic state from constant stress or other causes), the system can deliver high-frequency inhibitory therapy signals to decrease the activity or input to a particular organ or nerve structure and thereby effectively modulate, normalize, and optimize the ANS dysregulation responsible for fibromyalgia. In another example, a therapy delivery device of a closed-loop system can be activated at the onset of an episode (e.g., the onset of a sign and/or symptom) associated with fibromyalgia or, alternatively, the therapy delivery device can be activated continuously or intermittently to reduce or eliminate the frequency of such episode(s).

Methods

Another aspect of the present disclosure includes methods for treating fibromyalgia in a subject. In general, methods of the present disclosure can include the steps of: providing a therapy delivery device: placing the therapy delivery device into electrical communication with an ANS nerve target associated with fibromyalgia; and activating the therapy delivery device to deliver a therapy signal to the ANS nerve target in an amount and for a time sufficient to effect a change in sympathetic and/or parasympathetic activity in the subject and thereby treat the fibromyalgia. Subjects treatable by the present disclosure can, in some instances, have fibromyalgia as well as one or more related or unrelated medical conditions. Non-limiting examples of additional medical conditions can include morning stiffness, tingling or numbness in the hands and feet, headaches (e.g., migraines), irritable bowel syndrome, sleep disturbances, problems with thinking and memory (“fibre fog”), and painful menstrual periods.

In some instances, the step of placing a therapy delivery device into electrical communication with an ANS nerve target can entail different surgical and/or medical techniques, depending upon the ANS nerve target, for example, in some instances, a therapy delivery device can be surgically placed into electrical communication with an ANS nerve target via a percutaneous or endoscopic route. In other instances, a therapy delivery device can be placed into electrical communication with an ANS nerve target via an intravascular or intraluminal route. In further instances, a therapy delivery device can be placed into electrical communication with an ANS nerve target via a transcutaneous approach.

Examples of ANS nerve targets into which a therapy delivery device may be placed in electrical communication with can include, but are not limited to, a sympathetic chain ganglion, an efferent of a sympathetic chain ganglion, or an afferent of a sympathetic chain ganglion, in some instances, the sympathetic chain ganglion can be selected from the group consisting of a superior cervical ganglion, a middle cervical ganglion, an inferior cervical ganglion, and a stellate ganglion. Additional examples of ANS nerve targets into which a therapy delivery device may be placed into electrical communication can include one or more parasympathetic nerves or nerve structures. In one example, a therapy delivery device can be placed into electrical communication with a parasympathetic nerve or nerve structure-other than the vagus nerve.

In yet another example, a therapy delivery device can be configured for transcutaneous neuromodulation using magnetic stimulation. A magnetic stimulation device or system can generally include a pulse generator (e.g., a high current pulse generator) and a stimulating coil capable of producing magnetic pulses with desired field strengths. Other components of a magnetic stimulation device can include transformers, capacitors, microprocessors, safety interlocks, electronic switches, and the like. In operation, the discharge current flowing through the stimulating coil can generate the desired magnetic field or lines of force. As the lines of force cut through tissue (e.g., neural tissue), a current is generated in that tissue, if the induced current is of sufficient amplitude and duration such that the cell membrane is depolarized, nervous tissue will be stimulated in the same manner as conventional electrical stimulation. It is therefore worth noting that a magnetic field is simply the means by which an electrical current is generated within the nervous tissue, and that it is the electrical current, and not the magnetic field, which causes the depolarization of the cell membrane and thus stimulation of the target nervous tissue. Thus, in some instances, advantages of magnetic over electrical stimulation can include: reduced or sometimes no pain; access to nervous tissue covered by poorly conductive structures; and stimulation of nervous tissues lying deeper in the body without requiring invasive techniques or very high energy pulses.

Other examples of transcutaneous therapy delivery devices and systems that may be used as part of the present disclosure are described in U.S. Provisional Patent Application Ser. No. 61/693,946, filed Sep. 19, 2012, and No. 61/702,876, filed Aug. 28, 2012. It will be appreciated that transcutaneous therapy delivery devices and systems can additionally or optionally include any wearable item, accessory, article of clothing, or any object, device, or apparatus that a subject can use and, during use, comes into close or direct contact with a portion of the subject's body (e.g., the subject's neck). Examples of such transcutaneous neuromodulation devices can include vests, sleeves, shirts, socks, shoes, underwear, belts, scarves, wrist bands, gloves, ear pieces, band-aids, turtle neck, pendants, buttons, earrings, stickers, patches, bio-films, skin tattoos (e.g., using neuro-paint), chairs, computers, beds, head rests (e.g., of a chair or car seat), cell phones, and the like.

After placing the therapy delivery device, the therapy delivery device can be activated to deliver a therapy signal to the ANS nerve target and thereby treat fibromyalgia. In some instances, the therapy signal can include an electrical signal capable of electrically modulating the ANS nerve target. In one example, the therapy signal can include an electrical signal capable of electrically modulating at least a portion of the ANS. Electrical modulation of the ANS may affect central motor output, nerve conduction, neurotransmitter release, synaptic transmission, and/or receptor activation. For example, at least a portion of the ANS may be electrically modulated to alter, shift, or change parasympathetic activity from a first state to a second state, where the second state is characterized by an increase or decrease in parasympathetic activity relative to the first state. Alternatively, at least a portion of the ANS may be electrically modulated to alter, shift, or change sympathetic activity from, a first state to a second state, where the second state is characterized by an increase or decrease in sympathetic activity relative to the first state, it will be appreciated that delivering electrical energy, for example, to an ANS nerve target can modulate the ANS in any desirable combination of ways, such as increasing both parasympathetic and sympathetic activity, increasing parasympathetic activity while decreasing sympathetic function, decreasing both parasympathetic and sympathetic activity, and decreasing parasympathetic activity while increasing sympathetic activity.

In subjects with fibromyalgia as well as other conditions (e.g., hot flashes, CRPS, etc.), clinicians have used pharmacological blockade of the sympathetic nervous system, and in particular the stellate ganglion, to achieve some clinical benefit. Injection of local anesthetics into the stellate ganglion, however, presents a number of drawbacks, such as: a temporary effect that requires repeated injections; spread of the anesthetic to other important tissues, which can result in difficulty swallowing, vocal cord paralysis, and occasionally pneumothorax: and extreme difficultly in titrating the level of blockade. Advantageously, the present disclosure permits blockade, activation, and/or modulation of the ANS (e.g., the sympathetic ganglia) at the onset of symptomatic episodes associated with fibromyalgia and/or drive SNS and/or PNS activity during other periods to help reduce or prevent the frequency of such episodes. As discussed above, aspects of the present disclosure can be controllable (at least in part) by feedback from the subject as well as physiological parameters which are associated measures of the SNS and/or PNS response. Essentially all of the drawbacks associated with pharmacological blockade are overcome by the present disclosure as therapy delivery devices and/or related systems may be permanently implanted.

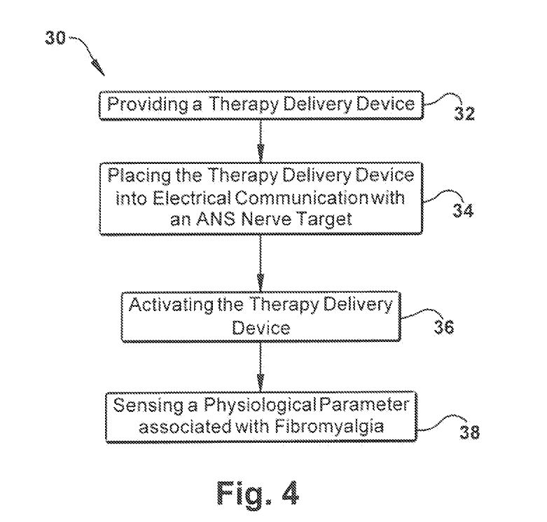

Another aspect of the present disclosure is illustrated in FIG. 4 and includes a method 30 tor treating fibromyalgia in a subject. Generally, the method 30 can include placing a therapy delivery device into electrical communication with an ANS nerve target associated with fibromyalgia (Steps 32-34). In one example, the method 30 can entail transvascular or transluminal delivery of one or more therapy signals to an ANS nerve target associated with fibromyalgia. Thus, in some instances, the method 30 can include providing a therapy delivery device configured for transvascular or transluminal insertion and placement within the subjectr. For instance, a therapy delivery device configured tor intravascular or intraluminal placement in a subject can include an expandable electrode as disclosed in the '331 application.

The therapy delivery device can be Inserted into a vessel or lumen of the mammal. Non-limiting examples of vessel and lumens into which the therapy delivery device can be inserted include arteries, veins, an esophagus, a trachea, a vagina, a rectum, or any other bodily orifice. The therapy delivery device can be surgically inserted into the vessel or lumen via a percutaneous, transvascular, laparoscopic, or open surgical procedure.

After inserting the therapy delivery device into the vessel or lumen, the therapy delivery device can be advanced (if needed) to an intraluminal target site of the ANS and placed into electrical communication therewith (Step 34). In some instances, advancement of the therapy delivery device can be done under image guidance (e.g., fluoroscopy, CT, MRI, etc.). Intraluminal target sites can include intravascular or intraluminal locations at which the therapy delivery device can he positioned. For example, an intraluminal target site can include a portion of a vessel wall that is innervated by (or in electrical communication with) a nerve, neuron, and/or nervous tissue of the ANS. Examples of intraluminal target sites can include, without limitation, vascular or luminal sites Innervated by and/or in electrical communication with neurons, axons, fibers, tracts, nerves, plexus, afferent plexus fibers, efferent plexus fibers, ganglion, pre-ganglionic fibers, postganglionic fibers, parasympathetic nerves and nerve structures (e.g., other than the vagus nerve), cervical sympathetic ganglia/ganglion, afferents thereof, efferents thereof, a sympathetic chain ganglion, an upper cervical chain ganglion, a lower cervical ganglion, an inferior cervical ganglion, a stellate ganglion, and a parasympathetic nerve or nerve structure.

The therapy delivery device can be activated to deliver a therapy signal to the intraluminal target site in an amount and for a time sufficient to effect a change in sympathetic and/or parasympathetic activity in the subject (Step 36). In some instances, fibromyalgia can be caused by, or associated with, hypersympathetic activity. In such instances, it may be desirable to block one or more SNS nerve targets (e.g., using ultrasound or radiofrequency energy) to decrease sympathetic activity in the subject. In other instances, it may be desirable to deliver a therapy signal (e.g., ultrasound or radio frequency) to one or more PNS nerve targets to increase parasympathetic activity in the subject.

In another aspect, the method 30 can include providing a therapy delivery device (Step 32) configured for placement and implantation within the subject. In one example, the therapy delivery device can comprise an electrode array configured for percutaneous implantation in the subject. The therapy delivery device can he placed into direct or indirect electrical contact with an ANS nerve target (Step 34), in some instances, “direct electrical contact” can mean that the therapy delivery device is placed on or in the ANS nerve target. In other instances, “indirect electrical contact” can mean that the therapy delivery device is located adjacent (but not in physical contact with) the ANS nerve target such that delivery of a therapy signal can modulate a function, activity, and/or characteristic of the ANS nerve target. Examples of ANS nerve targets arc listed above.

After placing the therapy delivery device into electrical contact with the ANS nerve target a therapy signal is delivered to the ANS nerve target (Step 36). The therapy signal can be delivered in an amount and for a time sufficient to effect a change in sympathetic and/or parasympathetic activity in the subject In one example, a therapy signal (e.g., ultrasound or radiofrequency energy) can be delivered to the stellate ganglion by an electrode or electrode array that is placed directly on or in the stellate ganglion. In some instances, fibromyalgia may be caused by hypersympathetic activity. In such instances, it may be desirable to deliver blocking stimulation to the stellate ganglion to decrease sympathetic activity and thereby normalize sympathetic activity in the subject. In other instances. It may be desirable to deliver a stimulatory signal to a PNS nerve target to increase parasympathetic activity in the subject.

In another aspect, the method 30 can include providing a therapy delivery device (Step 32) configured tor placement on the skin of the subject. Examples of therapy delivery devices configured for transcutaneous delivery of one or more therapy signals are described above. In some instances, the therapy delivery device can be positioned about the skin of the subject, without penetrating the skin of the subject, so that the therapy delivery device is in electrical communication with one or more ANS nerve targets associated with fibromyalgia (Step 34). Non-limiting examples of ANS nerve targets into which the therapy delivery device can be placed into electrical communication with are described above, in one example, the therapy delivery device can be positioned over a portion of the subject's skin, but without penetrating the skin, so that, at least a portion of the therapy delivery device overlies the ANS nerve target.

After appropriately positioning the therapy delivery device, a therapy signal can be delivered from the therapy delivery device to one or more ANS nerve targets associated with fibromyalgia (Step 36). The therapy signal can be delivered in an amount and for a time sufficient to effect a change in sympathetic and/or parasympathetic activity in the subject. In one example, a therapy signal (e.g., ultrasound or radiofrequency energy) can be transcutaneously delivered to the stellate ganglion. In some instances, fibromyalgia may he caused by hypersympathetic activity. In such instances, it may be desirable to deliver blocking stimulation to the stellate ganglion to decrease sympathetic activity and thereby normalize sympathetic activity in the subject. In other instances, it may be desirable to deliver a stimulatory signal to a PNS nerve target to increase parasympathetic activity in the subject.

Where the therapy delivery device is configured as a closed-loop system, it will be appreciated that the method 30 can additionally or optionally include sensing a physiological parameter (discussed above) associated with fibromyalgia (Step 38). For example, the level of one or more physiological parameters can be detected by a sensor (or sensors) disposed on or within the subject. A sensor signal, can then be generated based on the detected physiological parameter. Next, the therapy delivery device can be activated to adjust application of the therapy signal to the ANS nerve target in response to the sensor signal to treat, prevent, or mitigate fibromyalgia.

Another aspect of the present disclosure relates to a method for treating fibromyalgia by stimulation of one or more components of the central nervous system (CNS). One step of the method can include placing a therapy delivery device into electrical communication with a CNS nerve target associated with the fibromyalgia. Next, the therapy delivery device can be activated to deliver a therapy signal to the CNS nerve target in an amount and for a time sufficient to treat the fibromyalgia. Systems and devices for stimulating a CNS nerve target are described above and can include percutaneous devices, transvascular devices, and transdermal devices capable of delivering one or more therapy signals to the CNS nerve target. In one example, a CNS nerve target can include one or more regions of the brain which may targeted for stimulation using transcranial magnetic stimulation (IMS), in another example, a CNS nerve target can include one or more regions of the brain which may targeted for stimulation using deep brain, stimulation (DBS). In yet another example, a CNS nerve target can include spinal nervous tissue.

From the above description of the present disclosure, those skilled in the art will perceive improvements, changes and modifications. Such improvements, changes, and modifications are within the skill, of those in the art and are intended to be covered by the appended claims. All patents, patent applications, and publication cited herein are incorporated by reference in their entirety.

The following is claimed:

1. A method for treating fibromyalgia in a subject, the method comprising the steps of:
placing a therapy delivery device into electrical communication with a parasympathetic nervous system (PNS) nerve target, other than the vagus nerve, which is associated with the fibromyalgia; and
activating the therapy delivery device to deliver a therapy signal to the PNS nerve target in an amount and. for a time sufficient to effect a change in parasympathetic activity in the subject and thereby treat the fibromyalgia.

2. The method of claim 1, wherein the therapy signal is electrical energy,

3. The method of claim 1, wherein the therapy signal includes a non-burst stimulation pattern.

4. The method of claim 1, further comprising the steps of:
sensing a physiological parameter associated with the fibromyalgia;
generating a sensor signal based on the physiological parameter; and
activating the therapy delivery device to adjust application of the therapy signal to the PNS nerve target In response to the sensor signal to treat the fibromyalgia.

5. A method for treating fibromyalgia in a subject, said method comprising the steps of:
placing a therapy delivery device into electrical communication with an autonomic nervous system (ANS) nerve target associated with the fibromyalgia; and
activating the therapy delivery device to deliver an ultrasonic therapy signal to the ANS nerve target in an amount and for a time sufficient to effect a change in sympathetic and/or parasympathetic activity in the subject and thereby treat the fibromyalgia.

6. The method of claim 5, wherein said placing step further comprises directly contacting a portion of the therapy delivery device with the ANS nerve target.

7. The method of claim 5, wherein said placing step further comprises:
inserting the therapy delivery device into a blood vessel or lumen of the subject; and
advancing the therapy delivery device to a location substantially adjacent the ANS nerve target.

8. The method of claim 5, wherein said placing step further comprises positioning the therapy delivery device over a portion of the subject's skin, without penetrating the skin, so that at least a portion of the therapy delivery device overlies the ANS nerve target.

9. The method of claim 5, wherein the ANS nerve target is a PNS nerve target.

10. The method of claim 5, wherein the ANS nerve target is a sympathetic nervous system (SNS) nerve target.

11. The method of claim 10, wherein the SNS nerve target is a sympathetic nerve chain ganglion.

12. A method for treating fibromyalgia in a subject, said method comprising the steps of:
placing a therapy delivery device into electrical communication with an ANS nerve target associated with the fibromyalgia; and
activating the therapy delivery device to deliver a radiofrequency therapy signal to the ANS nerve target in an amount and for a time sufficient to effect a change in sympathetic and/or parasympathetic activity in the subject and thereby treat the fibromyalgia.

13. The method of claim 12, wherein said placing step further comprises directly contacting a portion of the therapy delivery device with the ANS nerve target.

14. The method of claim 12, wherein said placing step further comprises:
inserting the therapy delivery device into a blood vessel or lumen of the subject; and
advancing the therapy delivery device to a location substantially adjacent the ANS nerve target.

15. The method of claim 12, wherein said placing step further comprises positioning the therapy delivery device over a portion of the subject's skin, without penetrating the skin, so that at least a portion of the therapy delivery device overlies the ANS nerve target.

16. The method of claim 12, wherein the ANS nerve target is a PNS nerve target.

17. The method of claim 12, wherein the ANS nerve target is a SNS nerve target.

18. The method of claim 17, wherein the SNS nerve target is a sympathetic nerve chain ganglion.
